(19) United States
(12) Patent Application Publication
(10) Pub. No.: US 2020/0037850 A1
(43) Pub. Date: Feb. 6, 2020

(54) ENDOSCOPE BENDING PORTION, ENDOSCOPE INSERTION PORTION AND ENDOSCOPE

(71) Applicant: OLYMPUS CORPORATION, Tokyo (JP)

(72) Inventor: Nobuaki AKUI, Tokyo (JP)

(73) Assignee: OLYMPUS CORPORATION, Tokyo (JP)

(21) Appl. No.: 16/601,717

(22) Filed: Oct. 15, 2019

Related U.S. Application Data
(63) Continuation of application No. PCT/JP2018/014619, filed on Apr. 5, 2018.

(30) Foreign Application Priority Data
Apr. 17, 2017 (JP) 2017-081352

(51) Int. Cl. A61B 1/005

(52) U.S. Cl. CPC A61B 1/0057

(57) ABSTRACT

An endoscope bending portion is formed of a multilumen tube, and includes: two lumens through which a pair of bending operation wires for performing a bending operation in a first direction and in a second direction opposite to the first direction are individually inserted; and a bending direction restriction member configured to restrict bending in a direction of a second axis orthogonal to a first axis passing through a center axis in the first direction and the second direction.

### CROSS REFERENCE TO RELATED APPLICATION

This application is a continuation application of PCT/JP2018/014619 filed on Apr. 5, 2018 and claims benefit of Japanese Application No. 2017-081352 filed in Japan on Apr. 17, 2017, the entire contents of which are incorporated herein by this reference.

### BACKGROUND OF THE INVENTION

### 1. Field of the Invention

The present invention relates to an endoscope bending portion, an endoscope insertion portion and an endoscope in which a multilumen tube is used in at least a bending portion of an insertion portion.

### 2. Description of the Related Art

An endoscope includes an insertion portion that is formed in an elongated tubular shape according to an observation target and inserted into a lumen in order to cope with various observation targets. An endoscope having an insertion portion which is provided with a bending portion for varying an observation visual field direction is known.

Such an endoscope bending portion is disclosed in Japanese Patent Application Laid-Open Publication No. H08-94941, for example. In the endoscope bending portion in Japanese Patent Application Laid-Open Publication No. H08-94941, a multilumen tube is used as a bending member so that a diameter can be narrowed at a low cost.

### SUMMARY OF THE INVENTION

An endoscope bending portion according to an aspect of the present invention is the endoscope bending portion formed of a multilumen tube in which a plurality of hole portions are disposed so as to pass through in a longitudinal direction, and includes: two lumens through which a pair of bending operation wires for performing a bending operation in a first direction and in a second direction opposite to the first direction are individually inserted; and a bending direction restriction member configured to restrict bending in a direction of a second axis orthogonal to a first axis passing through a center axis in the first direction and the second direction.

An endoscope insertion portion according to an aspect of the present invention includes an endoscope bending portion formed of a multilumen tube in which a plurality of hole portions are disposed so as to pass through in a longitudinal direction, and the endoscope bending portion includes: two lumens through which a pair of bending operation wires for performing a bending operation in a first direction and in a second direction opposite to the first direction are individually inserted; and a bending direction restriction member configured to restrict bending in a direction of a second axis orthogonal to a first axis passing through a center axis in the first direction and the second direction.

In an endoscope according to an aspect of the present invention, an insertion portion is provided with an endoscope bending portion formed of a multilumen tube in which a plurality of hole portions are disposed so as to pass through in a longitudinal direction, the endoscope bending portion including: two lumens through which a pair of bending operation wires for performing a bending operation in a first direction and in a second direction opposite to the first direction are individually inserted; and a bending direction restriction member configured to restrict bending in a direction of a second axis orthogonal to a first axis passing through a center axis in the first direction and the second direction.

### BRIEF DESCRIPTION OF THE DRAWINGS

FIG. 1 is a general perspective view illustrating a schematic configuration of an endoscope of an aspect of the present invention;

FIG. 2 is a perspective view illustrating a configuration of a distal end part of an insertion portion;

FIG. 3 is a longitudinal sectional view of the distal end part of the insertion portion;

FIG. 4 is a perspective view illustrating a rigid portion provided in the insertion portion;

FIG. 5 is a V-V line sectional view of FIG. 4;

FIG. 6 is a partial sectional view illustrating the rigid portion provided in the insertion portion;

FIG. 7 is a sectional view illustrating the rigid portion provided in the insertion portion;

FIG. 8 is a side view illustrating a state where a bending portion of the insertion portion is bent in up and down directions;

FIG. 9 is a sectional view illustrating the rigid portion provided in the bending portion of a first modification;

FIG. 10 is a sectional view illustrating the rigid portion provided in the bending portion of a second modification;

FIG. 11 is a sectional view illustrating the rigid portion provided in the bending portion of a third modification;

FIG. 12 is a sectional view illustrating the rigid portion provided in the bending portion of a fourth modification;

FIG. 13 is a sectional view illustrating the rigid portion provided in the bending portion of a fifth modification;

FIG. 14 is a sectional view illustrating the rigid portion provided in the bending portion of a sixth modification; and

FIG. 15 is a perspective view illustrating the rigid portion provided in the bending portion of a seventh modification.

### DETAILED DESCRIPTION OF THE PREFERRED EMBODIMENT(S)

Hereinafter, a preferable embodiment of the present invention will be described with reference to the drawings. Note that, in the respective drawings used in the following description, a scale is made different for each component in order to illustrate the respective components in such sizes that the components can be recognized on the drawings, and the present invention is not limited only to quantities of the components, shapes of the components, ratios of the sizes of the components, and relative positional relationships of the respective components described in the drawings. In addition, in the following description, up and down directions in a view toward a paper surface of the drawing are sometimes described as an upper part and a lower part of the component.

First, an aspect of the present invention will be described below based on the drawings.

A schematic configuration of an endoscope of a first embodiment of the present invention will be described below based on the drawings.

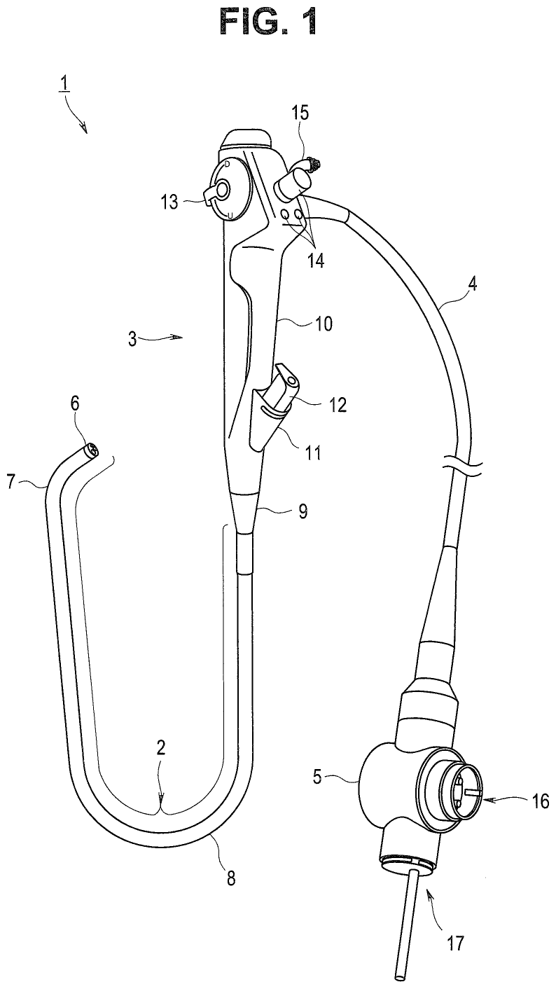

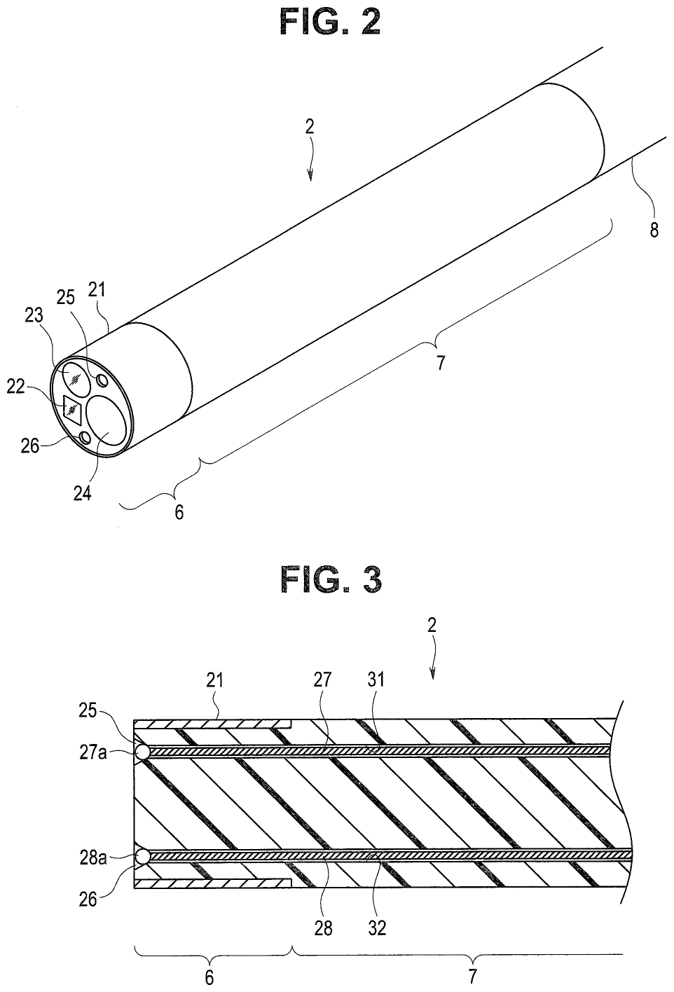

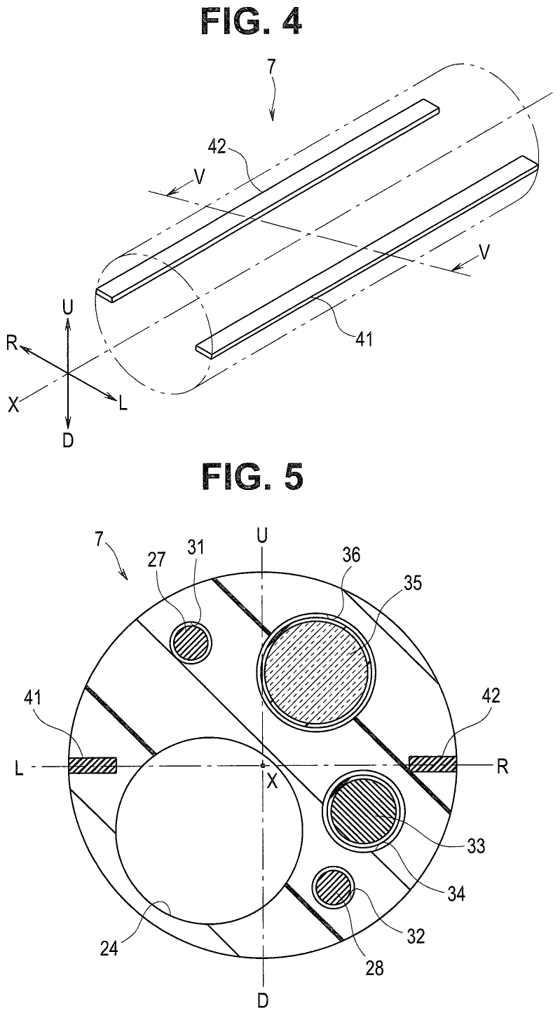

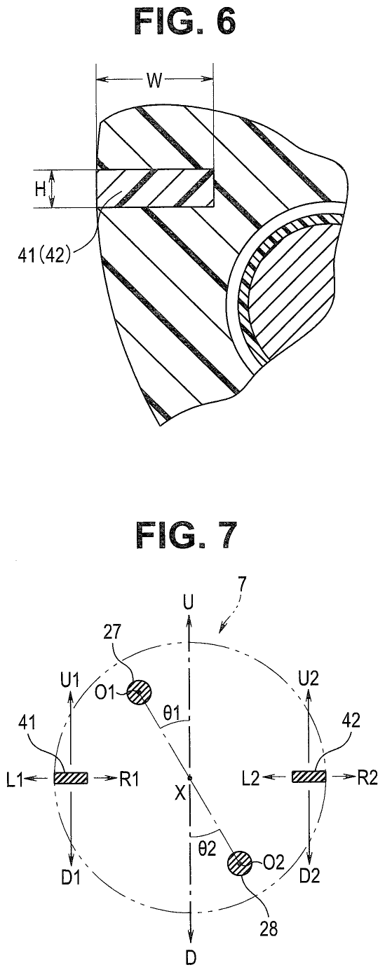

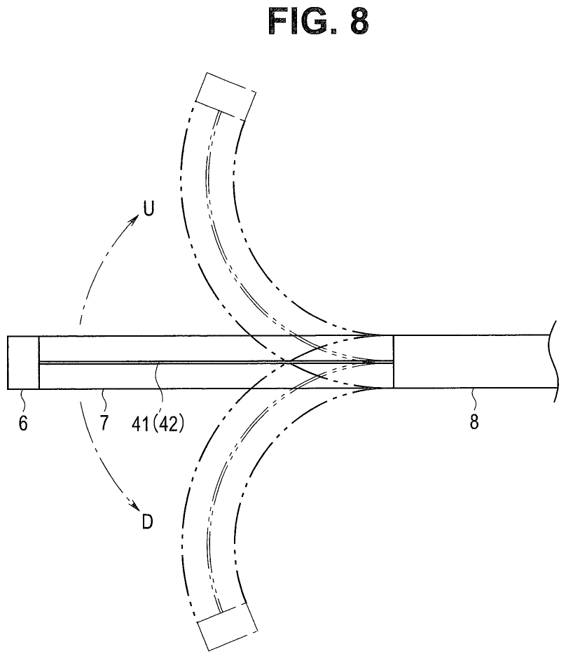

FIG. 1 is a general perspective view illustrating a schematic configuration of an endoscope of an aspect of the present invention, FIG. 2 is a perspective view illustrating a configuration of a distal end part of an insertion portion, FIG. 3 is a longitudinal sectional view of the distal end part of the insertion portion, FIG. 4 is a perspective view illustrating a rigid portion provided in the insertion portion, FIG. 5 is a V-V line sectional view of FIG. 4, FIG. 6 is a partial sectional view illustrating the rigid portion provided in the insertion portion, FIG. 7 is a sectional view illustrating the rigid portion provided in the insertion portion, and FIG. 8 is a side view illustrating a state where a bending portion of the insertion portion is bent in up and down directions.

An endoscope 1 of the first embodiment of the present invention is, as illustrated in FIG. 1, mainly configured by an insertion portion 2, an operation portion 3, a universal cord 4, an endoscope connector 5, and the like.

The whole insertion portion 2 is formed of a multilumen tube, here, which is formed in an elongated shape and is to be inserted into a subject. The insertion portion 2 is formed by connecting a distal end portion 6, a bending portion 7 that is an endoscope bending portion and a flexible tube portion 8 in order from a distal end side, and is flexible as a whole.

Note that the insertion portion 2 here is configured to be a single use (disposable) type by being fanned by the multilumen tube that can be manufactured at a low cost.

The distal end portion 6 of the insertion portion 2 incorporates an image pickup unit that is an image pickup apparatus including an image pickup device or the like inside and an illumination optical system configured to emit illumination light to front or the like (neither is illustrated).

Note that a form of the endoscope to which the present invention is applicable is not limited to an example described above (an electronic endoscope provided with an image pickup unit or the like), but may be other forms such as a so-called fiber scope in the form where the image pickup unit is not provided and an image guide fiber is disposed in the insertion portion 2.

The bending portion 7 is configured to receive a turning operation of a bending lever 13 for performing a bending operation among operation members provided in the operation portion 3 and be actively bendable in two (up and down) directions here in a first direction and a second direction which is the direction opposite to the first direction. Note that the up and down directions here are the up and down directions in an endoscope image picked up by the image pickup unit.

The flexible tube portion 8 is configured to have flexibility so as to be passively freely flexible. Inside the flexible tube portion 8, in addition to a lumen for a treatment instrument insertion channel, a lumen through which various kinds of signal lines are inserted, the signal lines being extended from the image pickup unit incorporated in the distal end portion 6 and extended through inside of the operation portion 3 to the inside of the universal cord 4, and a lumen through which a light guide (not illustrated) is inserted, the light guide guiding light emitted from a light source device (not illustrated) which is an external device to an illumination window (not illustrated) provided on a distal end face of the distal end portion 6, are formed.

Note that a light source may be in the form of providing a luminous body (a light emitting diode (LED), for example) inside the operation portion. In a case of the configuration, the light guide (not illustrated) is utilized to guide the light emitted from the LED inside the operation portion to the illumination window of the distal end portion 6.

Another form may be the form of providing the luminous body such as the LED inside the distal end portion 6, on a part near a proximal end of the illumination window, for example. In the case of the configuration, the light emitted from the LED is directly transmitted through the illumination window and illuminates the front of the distal end portion 6.

In other words, in the configuration, the light guide (not illustrated) inside the flexible tube portion 8 is not needed. On the other hand, a power supply line for making the LED provided in the distal end portion 6 emit the light or the like is inserted through the lumen inside the flexible tube portion 8.

The operation portion 3 is a configuration unit connected to a proximal end portion of the insertion portion 2 and configured to include a plurality of operation members or the like. The operation portion 3 is configured by a bend preventing portion 9, a grasping portion 10, the plurality of operation members (13 and 14 or the like), a treatment instrument insertion portion 11, a suction valve 15, and the like.

The bend preventing portion 9 is provided on a connection part of the distal end portion of the operation portion 3 and the proximal end portion of the flexible tube portion 8, and is a protective member for preventing the flexible tube portion 8 from being unnecessarily and suddenly bent when the endoscope 1 is used, by covering the proximal end portion of the flexible tube portion 8.

The grasping portion 10 is a housing portion configured to house various kinds of configuration members inside. The grasping portion 10 is connected to the bend preventing portion 9. The grasping portion 10 is a part to be held in hand and grasped by a user when the endoscope 1 is used.

The plurality of operation members are provided on an outer surface of the grasping portion 10, and are the members for operating various kinds of functions of the endoscope 1. Examples of the plurality of operation members are, in addition to the bending lever 13 for performing the bending operation in the up and down directions of the bending portion 7, the operation member configured to perform air/water feeding operations and a suction operation, and an operation member 14 for performing operations respectively corresponding to the image pickup unit, an illumination unit, and the like.

The treatment instrument insertion portion 11 is a configuration portion including a treatment instrument insertion opening (not illustrated) configured to insert various kinds of treatment instruments (not illustrated), and provided with a treatment instrument insertion passage communicated with the treatment instrument insertion channel inside the operation portion 3.

Note that to the treatment instrument insertion portion 11, a forceps plug 12 is disposed, the forceps plug 12 being a lid member configured to open and close the treatment instrument insertion opening and configured to be freely attachable and detachable (exchangeable) to/from the treatment instrument insertion portion 11. The suction valve 15 is a connection portion for connecting a suction pipeline with a non-illustrated suction device.

The universal cord 4 is a hollow tubular member that is flexible and extends from the operation portion 3. The universal cord 4 is a composite cable, the various kinds of signal lines inserted from the distal end portion 6 of the insertion portion 2 to the inside of the insertion portion 2 and extended through the inside of the operation portion 3, the light guide (not illustrated) from the light source device (not illustrated) which is the external device, an air/water feeding tube from an air/water feeding device (not illustrated) which is the external device, and the like being inserted to the inside.

The endoscope connector 5 is disposed at a distal end of the universal cord 4, and is a connection member for securing connection with the external device. The endoscope connector 5 includes, on a side face portion, an electric connector portion 16 configured to connect a signal cable connecting a video processor (not illustrated) which is the external device.

In addition, the endoscope connector 5 is configured to include a light guide bundle configured to connect the light source device (not illustrated) which is the external device, a light source connector portion 17 configured to connect an electric cable (not illustrated) in which the various kinds of signal lines are gathered, and the like.

Next, the configuration of mainly the distal end part of the insertion portion 2 of the endoscope 1 will be described in detail below.

As illustrated in FIG. 2, in the insertion portion 2, an annular distal end main body tube 21 is extrapolated and fixed to the part configuring the distal end portion 6.

On the distal end face of the distal end portion 6, an observation window 22, an illumination window 23, an opening portion of a treatment instrument insertion channel 24, and two locking holes 25 and 26 configured to respectively lock distal ends of bending operation wires to be described later, configured to bend the bending portion 7, are disposed. On the proximal end side of the distal end portion 6, the bending portion 7 and the flexible tube portion 8 are disposed in order so as to be connected.

In the present embodiment, the distal end portion 6, and bending portion 7 and the flexible tube portion 8 are formed of an integrally-formed multilumen tube. For the multilumen tube configuring the insertion portion 2, for example, the multilumen tube formed by using a flexible material such as expanded PTFE (ePTFE) formed by expanding PTFE (polytetrafluoroethylene) which is one of fluororesins or various kinds of biocompatible elastomers is applied.

Note that the flexible tube portion 8 may be configured to apply a mesh-like tube to a spiral tube and apply a flexible tube to an outer periphery of the mesh-like tube. In the insertion portion 2, the plurality of lumens are bored to pass through, the lumens being communicated with the above-described, non-illustrated image pickup unit disposed inside the distal end portion 6, an image pickup cable of the image pickup unit, the illumination optical system, the light guide disposed on the proximal end side from the illumination optical system, the treatment instrument insertion channel 24 and the locking holes 25 and 26 for inserting the bending operation wires (described later).

As illustrated in FIG. 3, in the insertion portion 2 of the multilumen tube, a pair of wire insertion lumens 31 and 32 for respectively inserting two bending operation wires 27 and 28 configured to bend the bending portion 7 in the up and down directions are bored so as to pass through in a longitudinal axis direction.

Note that on the distal ends of the bending operation wires 27 and 28, locking pieces 27a and 28a are respectively provided. The locking pieces 27a and 28a are introduced into the locking holes 25 and 26 formed at a distal end face part of the distal end portion 6, locked, and joined and fixed inside the locking holes 25 and 26.

In other words, the locking holes 25 and 26 have a cone shape, an outer diameter of which increases toward the distal end face of the distal end portion 6 so that the respective locking pieces 27a and 28a are stuck and locked.

In the bending portion 7 of the present embodiment, as illustrated in FIG. 4 and FIG. 5, rigid portions 41 and 42 that are resin members formed of a rigid grade of being less flexible and more rigid than the material of the multilumen tube configuring the insertion portion 2, and are belt-like members as a plurality of, two in this case, bending direction restriction members are provided side by side in left and right directions (L-R) in a third direction orthogonal to the up and down directions (U-D) and a fourth direction which is the direction opposite to the third direction along a center axis X to be a longitudinal axis.

In other words, the rigid portions 41 and 42 are arranged along an axis (L-R) in the left and right directions passing through the center axis X in the bending portion 7, and are provided on both end parts of the outer periphery of the bending portion 7 to be point symmetrical positions with the center axis X as a point.

Note that the rigid portions 41 and 42 may be integrally provided in the bending portion 7 by extrusion molding when the multilumen tube is manufactured, or groove portions may be formed at the part to be the bending portion 7 and the rigid portions 41 and 42 may be assembled later by joining fixation or the like after multiple lumens are molded.

In the bending portion 7, in addition to the lumen configuring the treatment instrument insertion channel 24 described above and the pair of wire insertion lumens 31 and 32 to which the bending operation wires 27 and 28 are inserted, a cable insertion lumen 34 to which an image pickup cable 33 of the non-illustrated image pickup unit is inserted and a light guide insertion lumen 36 to which a light guide 35 is inserted are bored so as to pass through along the center axis X.

Note that the rigid portions 41 and 42 have, as illustrated in FIG. 6, a belt shape with a rectangular cross section, a height H of which is shorter than a width W, and are provided side by side such that the direction of the width W is along the axis (L-R) in the left and right directions.

As illustrated in FIG. 7, the two belt-like rigid portions 41 and 42 which are the bending direction restriction members generate large resistance to bending in the left and right directions (L1-R1, L2-R2) and thus restrict the bending in the left and right directions (L-R), and the bending portion 7 configured as above becomes easy to bend in the up and down directions (U-D).

In other words, since the width W in the left and right directions (L1-R1, L2-R2) is longer than the height H in the up and down directions (U1-D1, U2-D2), the bending in the left and right directions (L1-R1, L2-R2) is restricted, and the two rigid portions 41 and 42 become easy to bend in the up and down directions (U1-D1, U2-D2).

By providing the direction of the width W of the two rigid portions 41 and 42 in the left and right directions (L-R) in the bending portion 7 and on an axis (L-R) in the left and right directions passing through the center axis X bisecting the bending portion 7 up and down, the bending portion 7 can be configured to easily bend in the up and down directions (U-D).

Thus, without inserting centers 01 and 02 of the two bending operation wires 27 and 28 for bending the bending portion 7 in the up and down directions (U-D) so as to pass on the axis in the up and down directions (U-D) of the bending portion 7, as illustrated in FIG. 8, the bending portion 7 bends in the up and down directions.

In other words, even when the wire insertion lumens 31 and 32 are bored such that the centers 01 and 02 of the two bending operation wires 27 and 28 pass through positions rotated by predetermined angles 01 and 02 around the center axis X from the axis (U-D) in the up and down directions of the bending portion 7, the bending portion 7 can be bent in the up and down directions (U-D).

Note that one bending operation wire 27 is inserted to an upper side of the axis (L-R) in the left and right directions of the bending portion 7, and the other bending operation wire 28 is inserted to a lower side of the axis (L-R) in the left and right directions of the bending portion 7. It is preferable to bore the wire insertion lumens 31 and 32 such that the two bending operation wires 27 and 28 are inserted near the axis (U-D) in the up and down directions.

In this way, by making it possible to bend the bending portion 7 in the up and down directions even when boring parts of the wire insertion lumens 31 and 32 to insert the two bending operation wires 27 and 28 that bend the bending portion 7 of the insertion portion 2 formed of the multilumen tube in the up and down directions at different positions not coinciding with the up and down directions to bend the bending portion 7, the endoscope 1 of the present embodiment can be configured to increase a degree of freedom of a sectional layout of various kinds of internal components inserted to the insertion portion 2 and make a diameter of the insertion portion 2 narrower. Further, in the endoscope 1 of the present embodiment capable of making the diameter of the insertion portion 2 narrower, the configuration is effective for a urological endoscope, in particular.

### (First Modification)

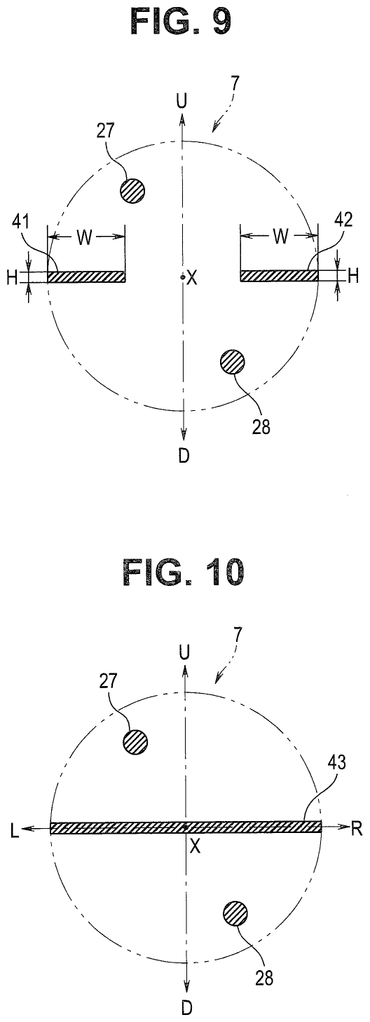

FIG. 9 is a sectional view illustrating the rigid portion provided in the bending portion of the first modification.

As illustrated in FIG. 9, by prolonging the width W of the two belt-like rigid portions 41 and 42 which are the bending direction restriction members and generating larger resistance to the bending in the left and right directions (L-R), the bending portion 7 may be made to easily bend in the up and down directions (U-D).

### (Second Modification)

FIG. 10 is a sectional view illustrating the rigid portion provided in the bending portion of the second modification.

As illustrated in FIG. 10, a belt-like rigid portion 43 which is the bending direction restriction member may be provided along the axis (L-R) in the left and right directions of the bending portion 7 so as to bisect the bending portion 7 up and down (U-D).

### (Third Modification)

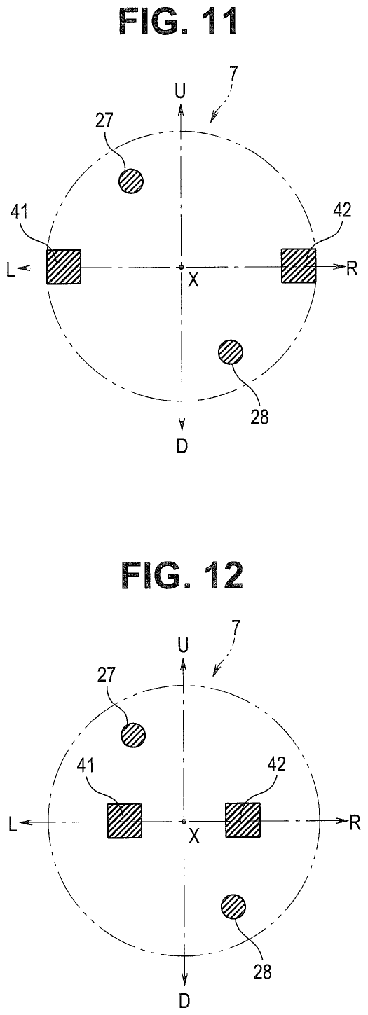

FIG. 11 is a sectional view illustrating the rigid portion provided in the bending portion of the third modification.

As illustrated in FIG. 11, the two belt-like rigid portions 41 and 42 which are the bending direction restriction members may have not the rectangular cross section but a square cross section, and the bending portion 7 can also be made to easily bend in the up and down directions (U-D) by providing the two rigid portions 41 and 42 side by side along the axis (L-R) in the left and right directions of the bending portion 7.

In other words, when the two rigid portions 41 and 42 are arranged as illustrated in FIG. 11, a deformation amount of the two rigid portions 41 and 42 needed when bending the bending portion in the left and right directions (L-R) is larger than the deformation amount needed when bending the bending portion in the up and down directions (U-D), and thus bending resistance in the left and right directions (L-R) becomes larger than bending resistance in the up and down directions (U-D). That is to say, the bending portion 7 becomes easy to bend in the up and down directions (U-D).

### (Fourth Modification)

FIG. 12 is a sectional view illustrating the rigid portion provided in the bending portion of the fourth modification.

As illustrated in FIG. 12, the two belt-like rigid portions 41 and 42 which are the bending direction restriction members may not be provided on both end parts of the outer periphery of the bending portion 7 but may be provided inside the bending portion 7.

Note that while the two rigid portions 41 and 42 having the square cross section are illustrated here, the two rigid portions 41 and 42 may of course have the rectangular cross section.

### (Fifth Modification)

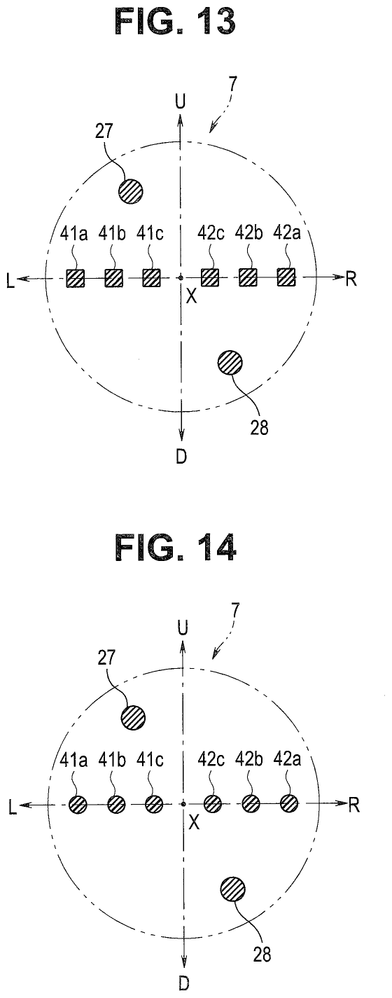

FIG. 13 is a sectional view illustrating the rigid portion provided in the bending portion of the fifth modification.

As illustrated in FIG. 13, belt-like rigid portions 41a, 41b, 41c, 42a, 42b and 42c which are the bending direction restriction members may not be provided on both end parts of the outer periphery of the bending portion 7 but may be provided in plurality, a total of six pieces here, inside the bending portion 7.

Note that while the six rigid portions 41a, 41b, 41c, 42a, 42b and 42c having the square cross section are illustrated here, the six rigid portions 41a, 41b, 41c, 42a, 42b and 42c may of course have the rectangular cross section.

### (Sixth Modification)

FIG. 14 is a sectional view illustrating the rigid portion provided in the bending portion of the sixth modification.

The rigid portions 41a, 41b, 41c, 42a, 42b and 42c which are the bending direction restriction members may not be in a rectangular shape like the rectangular cross section or the square cross section, but as illustrated in FIG. 14, may have a perfect circle cross section, for example, and be arranged along the axis (L-R) in the left and right directions of the bending portion 7 such that the bending resistance in the left and right directions (L-R) becomes larger than the bending resistance in the up and down directions (U-D), and the bending portion 7 may be made to easily bend in the up and down directions (U-D).

### (Seventh Modification)

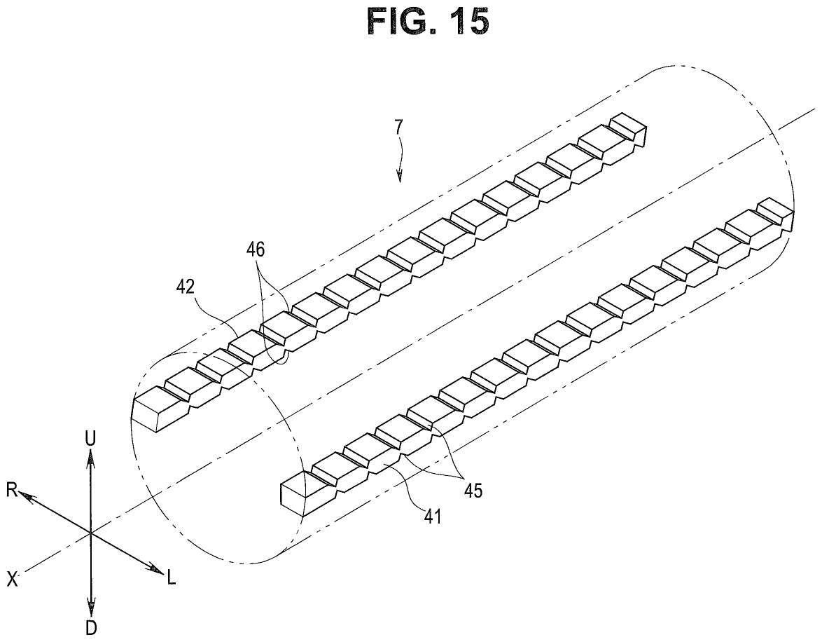

FIG. 15 is a perspective view illustrating the rigid portion provided in the bending portion of the seventh modification.

As illustrated in FIG. 15, the two rigid portions 41 and 42 which are the bending direction restriction members may be made to more easily bend in the up and down directions (U-D) by forming a plurality of groove portions 45 and 46 in the direction orthogonal to the longitudinal direction on upper and lower surfaces, and the bending portion 7 may be made to more easily bend in the up and down directions (U-D). V-shaped grooves are illustrated for the plurality of groove portions 45 and 46 here. However, the groove portions 45 and 46 may be U-shaped grooves, for example.

Note that in the embodiment and the respective modifications described above, the configuration in which the rigid portions 41 and 42 (43, 41a, 41b, 41c, 42a, 42b, 42c) which are the bending direction restriction members are provided only in the bending portion 7 is illustrated. However, the endoscope is not limited to this configuration, and the bending direction restriction members may be provided in an entire area of the insertion portion 2.

Further, it is preferable that the rigid portions 41 and 42 (43, 41a, 41b, 41c, 42a, 42b, 42c) which are the bending direction restriction members are provided on the axis (L-R) in the left and right directions passing through the center axis X of the bending portion 7. However, as long as the rigid portions are provided on the axis orthogonal to the axis (U-D) in the up and down directions of the bending portion 7, the bending in the left and right directions (L-R) of the bending portion 7 is restricted, and the bending portion 7 can be made to easily bend in the up and down directions (U-D).

The present invention is not limited to the embodiment described above, but of course various modifications and applications can be implemented without departing from a subject matter of the present invention. Further, the embodiment described above includes inventions in various stages, and various inventions can be extracted by appropriate combinations of a plurality of disclosed constituent elements.

For example, even when some constituent elements are deleted from the entire constituent elements indicated in the embodiment described above, in the case where a problem to be solved by the invention can be solved and effects of the invention can be obtained, the configuration from which the constituent elements are deleted can be extracted as the invention. Further, components over different embodiments may be appropriately combined. The present invention is not constrained by a specific embodiment of the invention other than being limited by an attached claim.

The present invention can realize an endoscope bending portion and an endoscope capable of eliminating need of aligning a position to insert the bending operation wire in a bending direction, improving the degree of freedom of a layout in a sectional direction of arranging various kinds of components, and making the diameter of the insertion portion narrower.

What is claimed is:

1. An endoscope bending portion formed of a multilumen tube in which a plurality of hole portions are disposed so as to pass through in a longitudinal direction, the endoscope bending portion comprising:
two lumens through which a pair of bending operation wires for performing a bending operation in a first direction and in a second direction opposite to the first direction are individually inserted; and
a bending direction restriction member configured to restrict bending in a direction of a second axis orthogonal to a first axis passing through a center axis in the first direction and the second direction.

2. The endoscope bending portion according to claim 1,
wherein the bending direction restriction member is provided on the second axis passing through the center axis.

3. The endoscope bending portion according to claim 1,
wherein the bending direction restriction member is provided in plurality, and
the plurality of bending direction restriction members are provided side by side on the second axis.

4. The endoscope bending portion according to claim 3,
wherein the bending direction restriction members are provided on both end parts of the multilumen tube along the second axis.

5. The endoscope bending portion according to claim 3,
wherein the bending direction restriction members are provided in point symmetrical positions with the center axis as a point.

6. The endoscope bending portion according to claim 1,
wherein the bending direction restriction member is a belt-like member, a length of which along the second axis is longer than a length along the first axis.

7. The endoscope bending portion according to claim 1,
wherein the lumens through which the bending operation wires are inserted are formed at positions different from positions on the first axis.

8. An endoscope insertion portion including the endoscope bending portion according to claim 1.

9. An endoscope in which an insertion portion is provided with the endoscope bending portion according to claim 1.
